(19) United States
(12) Patent Application Publication
(10) Pub. No.: US 2026/0269085 A1
(43) Pub. Date: Sep. 10, 2026

(54) INFUSION PUMP SYSTEM AND METHOD WITH MULTIPLE DRUG LIBRARY EDITOR SOURCE CAPABILITY

(71) Applicant: ICU Medical, Inc., San Clemente, CA (US)

(72) Inventors: James D. Jacobson, Lindenhurst, IL (US); Anatoly S. Belkin, Glenview, IL (US)

(21) Appl. No.: 19/664,302

(22) Filed: Apr. 30, 2026

Related U.S. Application Data
(62) Division of application No. 18/181,348, filed on Mar. 9, 2023, now Pat. No. 12,640,275.
Application No. 18/181,348 is a continuation of application No. 15/164,251, filed on May 25, 2016, now Pat. No. 11,605,468.
(60) Provisional application No. 62/166,381, filed on May 26, 2015.

(51) Int. Cl. G16H 70/40; A61M 5/172; G16H 20/17; G16H 40/40

(52) U.S. Cl. CPC G16H 70/40; A61M 5/172; G16H 20/17; G16H 40/40; A61M 2205/52

(57) ABSTRACT

An infusion pump system and method with multiple drug library editor source capability including: an infusion pump system having a first drug library editor operable to generate a first drug library; a second drug library editor operable to generate a second drug library; and an infusion pump operable to connect to either one of the first drug library editor and the second drug library editor, the infusion pump having an operational drug library being one of the first drug library received from the first drug library editor and the second drug library received from the second drug library editor. The first drug library editor is one of a dedicated drug library editor and an enterprise drug library editor, and the second drug library editor is the other of the one of the dedicated drug library editor and the enterprise drug library editor.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

### Field of the Invention

The present invention relates to medical devices. More specifically, the invention relates to infusion pump systems and methods with multiple drug library editor source capability.

### Description of the Related Art

Infusion pumps are medical devices that deliver fluids, including nutrients and medications such as antibiotics, chemotherapy drugs, and pain relievers, into a patient's body in controlled amounts. Many types of pumps, including large volume, patient-controlled analgesia (PCA), elastomeric, syringe, enteral, and insulin pumps, are used worldwide in healthcare facilities, such as hospitals, and in the home. Clinicians and patients rely on pumps for safe and accurate administration of fluids and medications.

More than 90 percent of surgical patients and one-third of non-surgical patients receive some form of intravenous therapy while in the hospital. Industry reports indicate an estimated 400,000 drug related injuries occur in hospitals annually generating $3.5 billion in extra medical costs. Safety software, including drug error reduction systems (DERS), has been developed to reduce medication errors, enhance quality care, and improve workflow.

Drug error reduction systems (DERS) typically use a drug library editor (DLE) to develop drug libraries including protocols, rule sets, and/or pump configuration settings, which are then loaded onto an infusion pump. The drug library at the infusion pump provides medication pick lists for the caregiver to select the desired therapy, medication profiles including hard and soft limits, and pump configuration settings including but not limited to distal pressure occlusion limits, air-in-line limits, callback settings, backlight display settings, etc.

Drug library editors are currently one of two types: dedicated DLEs or enterprise DLEs. Dedicated DLEs can only be connected to one infusion pump at a time to download the drug library to the individual infusion pump. Enterprise DLEs are part of an integrated healthcare facility system and can be connected to download a drug library to a number of infusion pumps.

Dedicated DLEs for a single type of infusion pump can implement drug error reduction systems in a simple cost-effective manner, but typically have reduced functionality when compared to enterprise DLEs. Dedicated DLEs generally provide protocol based libraries, in which the point of care caregiver selects a predefined protocol from a list, then accepts or modifies the predefined infusion parameters for the infusion to be administered. Dedicated DLEs work well for home, nursing homes, and other smaller facilities in which a limited number of medications are used.

Enterprise DLEs for multiple infusion pumps can implement drug error reduction systems as part of more complex and expensive systems having large drug libraries, rich functionality, and sophisticated reporting tools. Enterprise DLEs can be used to provide drug profile-based libraries, with the point of care caregiver selecting a medication from a medication list, then programming the infusion to be administered under limits assigned to the medication. Enterprise DLEs work well for hospitals, treatment centers, surgery centers, and other large facilities.

Unfortunately, no infusion pump is presently available which is able to obtain drug libraries from different types of drug library editors, e.g., from both dedicated DLEs and enterprise DLEs. Although in the case of enterprise DLEs, one DLE may be used with different types of infusion pumps, no infusion pump can receive drug libraries from more than one DLE even though one infusion pump type can be used in different settings, such as the home or hospital. In present practice, one infusion pump can receive a drug library from one specified type of drug library editor, but no other. For example, one infusion pump can receive a drug library from an enterprise drug library editor, but not a dedicated drug library editor. Similarly, a different kind of infusion pump can receive a drug library from a dedicated drug library editor, but not an enterprise drug library editor. Thus, the manufacturer must produce and the users must select different infusion pumps and associated DLEs for different applications, increasing cost and complexity. Different programming is required for dedicated DLEs and enterprise DLEs, increasing the confusion and effort required for programmers of different DLEs.

Healthcare systems which cover multiple levels of care from home care to hospital care must maintain different DLEs for different levels, increasing costs to the healthcare system and to the consumers.

It would be desirable to have infusion pump systems and methods with multiple drug library editor source capability that would overcome the above disadvantages.

### SUMMARY OF THE INVENTION

One aspect of the present invention provides an infusion pump system including a first drug library editor operable to generate a first drug library; a second drug library editor operable to generate a second drug library; and an infusion pump operable to connect to either one of the first drug library editor and the second drug library editor, the infusion pump having an operational drug library being one of the first drug library received from the first drug library editor and the second drug library received from the second drug library editor. The first drug library editor is one of a dedicated drug library editor and an enterprise drug library editor, and the second drug library editor is the other of the one of the dedicated drug library editor and the enterprise drug library editor.

Another aspect of the present invention provides a method of configuring one or more infusion pumps including providing an infusion pump having an operational drug library; preparing a first drug library on a first drug library editor; loading the first drug library into the infusion pump from the first drug library editor as the operational drug library; delivering an infusion from the infusion pump in accordance with the first drug library; preparing a second drug library on a second drug library editor; loading the second drug library into the infusion pump from the second drug library editor as the operational drug library; and delivering an infusion from the infusion pump in accordance with the second drug library. The first drug library editor is one of a dedicated drug library editor and an enterprise drug library editor, and the second drug library editor is the other of the one of the dedicated drug library editor and the enterprise drug library editor.

Another aspect of the present invention provides a method of configuring one or more infusion pumps including providing an infusion pump adapted to receive a drug library from one of multiple drug library editors and having an operational drug library; preparing one of a first drug library on a first drug library editor or a second drug library on a second drug library editor.

selecting one of the first drug library editor and the second drug library editor as a source for a received drug library; loading the received drug library into the infusion pump as the operational drug library; and delivering an infusion from the infusion pump in accordance with the operational drug library, wherein the first drug library editor is one of a dedicated drug library editor and an enterprise drug library editor, and the second drug library editor is the other of the one of the dedicated drug library editor and the enterprise drug library editor.

Yet another aspect of the present invention provides an infusion pump for selectable use with either one of a first drug library editor operable to generate a first drug library and a second drug library editor operable to generate a second drug library, the first drug library editor being one of a dedicated drug library editor and an enterprise drug library editor, and the second drug library editor being the other of the one of the dedicated drug library editor and the enterprise drug library editor. The infusion pump includes a memory operable to store an operational drug library; a flow controller operably connected to the memory; and a fluid driver operably connected to the flow controller. The memory is operable to receive a selection of one of the first drug library from the first drug library editor and the second drug library from the second drug library editor as the operational drug library, and the flow controller is operable to control the fluid driver in accordance with the received operational drug library.

The foregoing and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is a block diagram of an infusion pump system with multiple drug library editor source capability in accordance with the present invention;

FIG. 2 is a block diagram of a medication management system with an enterprise drug library editor for an infusion pump system with multiple drug library editor source capability in accordance with the present invention.

FIG. 3 is a block diagram of a dedicated infusion pump system with a dedicated drug library editor for an infusion pump system with multiple drug library editor source capability in accordance with the present invention.

FIG. 4 is a block diagram of an infusion pump with multiple drug library editor source capability in accordance with the present invention;

FIG. 5 is a flowchart of a method of use for an infusion pump system with multiple drug library editor source capability in accordance with the present invention.

FIG. 6 is a flowchart of another method of use for an infusion pump system with multiple drug library editor source capability in accordance with the present invention.

Like elements share like reference numbers throughout the various figures.

### DETAILED DESCRIPTION OF THE PREFERRED EMBODIMENT

An infusion pump system with multiple drug library editor source capability is described herein. The infusion pump can receive an operational drug library from either one of a dedicated drug library editor or an enterprise drug library editor. Thus, the infusion pump can be used with a medication management system such as used in a large enterprise medical care facility like a hospital or in a small dedicated care facility such as a nursing home or the home of a patient.

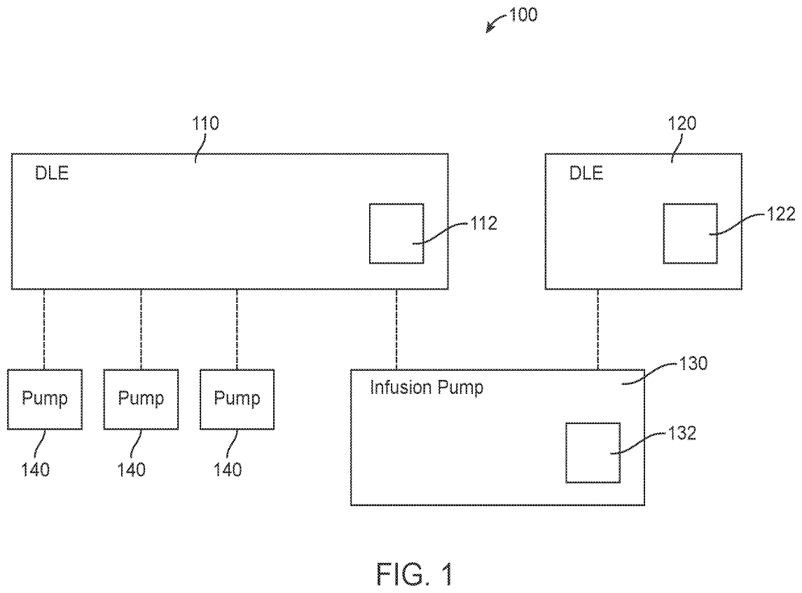

FIG. 1 is a block diagram of an infusion pump system with multiple drug library editor source capability in accordance with the present invention. In this example, the infusion pump system includes a dedicated drug library editor and an enterprise drug library editor. The infusion pump provides a therapeutic fluid from a fluid reservoir to a patient in accordance with operating parameters and/or limits established in an operational drug library.

An infusion pump system 100 includes a first drug library editor (DLE) 110, a second drug library editor (DLE) 120, and an infusion pump 130. The first drug library editor 110 is operable to generate a first drug library 112 and the second drug library editor 120 is operable to generate a second drug library 122. The infusion pump 130 is operable to connect to either one of the first drug library editor 120 or the second drug library editor 130 as desired for a particular application. The infusion pump 130 has an operational drug library 132, which is one of the first drug library 112 received from the first drug library editor 110 or the second drug library 122 received from the second drug library editor 120. The first drug library editor 110 is one of a dedicated drug library editor and an enterprise drug library editor, and the second drug library editor 120 is the other of the one of the dedicated drug library editor and the enterprise drug library editor. Thus, one drug library editor is of one type and the other drug library editor is of a different type.

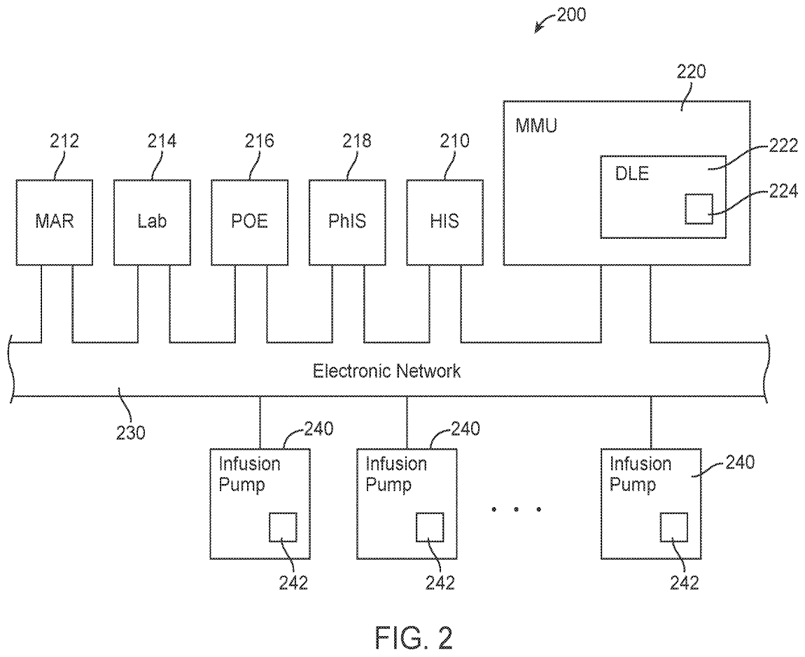

In this example, the first drug library editor 110 is the enterprise drug library editor and the second drug library editor 120 is the dedicated drug library editor. The first drug library editor 110 can provide operational drug libraries to a number of infusion pumps 140 in addition to the infusion pump 130. The enterprise drug library editor 110 is part of a medication management unit 220 that is in electronic communication or networked with other electronic information systems within a healthcare system, which facilitates or provides bi-directional communication between the pumps and the other electronic information systems, as best seen in FIG. 2. The second drug library editor 120 only provides drug libraries to one infusion pump at a time, in this case infusion pump 130. The dedicated drug library editor 120 as defined herein is a standalone drug library editor that is not networked to other information systems within a healthcare system. The dedicated drug library editor is operably connectable to and provides a drug library for a single type of infusion pump, typically a limited set of local infusion pumps such as one to fifteen infusion pumps in a single geographic location. The dedicated drug library editor is also limited in the functions it performs and typically only handles the creation, editing and downloading of a drug library to the pump. The enterprise drug library editor as defined herein is a drug library editor operably connectable to and providing drug libraries for a number of different types of infusion pumps, for example a multitude (more than fifteen) geographically dispersed pumps of various types (PCA, syringe, large volume parenteral, etc.). The enterprise drug library editor is typically a part of a larger suite of software applications run on or connected through a client computer to a server that has more functions such as bi-directional communications between the pump and server, as well as sophisticated logging and reporting on pump programming, user input, and operation (including but not limited to alarms, alerts, messages, overrides, program modifications, etc.). The application of the dedicated drug library editor and the enterprise drug library editor are described below in association with FIGS. 2 & 3, respectively.

Referring to FIG. 1, the first drug library editor 110 and second drug library editor 120 are operably connectable to the infusion pump 130 as indicated by the dashed lines between the drug library editors 110, 120 and the infusion pump 130. The connections between the drug library editors 110, 120 and the infusion pump 130 can be wired, wireless, or a combination of wired and wireless. For the enterprise drug library editor, the connections between the drug library editors 110, 120 and the infusion pump 130 can include intermediate components as desired for a particular application. One of the drug library editors 110, 120 can be connected to the infusion pump 130 to transfer the drug library 112, 122, respectively, to the infusion pump 130 as the operational drug library 132, and then the connection between the drug library editor and the infusion pump can be disconnected.

The first drug library editor 110 and second drug library editors 120 are used to create and/or modify the first drug library 112 and the second drug library 122, respectively. The drug library editor operates on the drug library within the drug library editor to support, maintain, edit, and/or export the operational drug library received by the infusion pump. A programmer (not shown), such as a licensed pharmacist, doctor, biomedical engineer, or the like, working with the drug library editor on a user interface can create and/or modify the drug libraries as desired for a particular application. The drug library editor can be deployed in a medication management system on one or more computers and enables the programmer to import, export, and edit whole drug libraries and individual drug library values to control and customize a drug library according to hospital preferences. In one example, drug libraries in the drug library editor can be formulated and edited using a conventional database management software platform, such as SQL Server or SQL Desktop Engine by Microsoft of Redmond, Washington.

The first drug library 112 and the second drug library 122 can each be part of a customizable drug library database available to the first drug library editor 110 and second drug library editors 120, respectively. The drug library includes drug and infusion pump related information, which may include but is not limited to drug name, drug class, drug concentration, drug amount, drug units, diluent amount, diluent units, dosing units, delivery dose or rate, medication parameters or limits (hard or soft), device/infuser settings and/or modes, clinical care area (CCA) designations and constraints, library version, and the like. The drug library can also include operating parameters for reporting from each of the infusion pumps back to the medication management system.

FIG. 2 is a block diagram of a medication management system 200 with an enterprise drug library editor for an infusion pump system with multiple drug library editor source capability in accordance with the present invention. The enterprise drug library editor is a drug library editor operably connectable to and providing drug libraries for a multitude of infusion pumps, for example more than fifteen pumps. One example of an enterprise drug library editor 222 is the Hospira MedNet™ Meds™ drug library editor from Hospira, Inc. of Lake Forest, Ill., U.S.A. The infusion pumps served by the enterprise drug library editor are typically of various different types. Types in this context can mean different operating mechanisms, manufacturers, models, or software or hardware versions, by way of example and not limitation.

The medication management system 200 provides an integrated enterprise-wide medication management system that reduces the risks of medication error and improves patient safety. The medication management system (MMS) 200 includes a hospital information system (HIS) 210, a medication management unit (MMU) 220, an electronic network 230, and infusion pumps 240. In one embodiment, the medication management system (MMS) 200 operates in a hospital environment which can be construed broadly as used herein to mean any medical care facility, including but not limited to a hospital, treatment center, clinic, doctor's office, day surgery center, hospice, nursing home, and any of the above associated with a home care environment. In one embodiment, the medication management system 200 operates in a large medical care facility, such as a hospital or treatment center. The medication management unit 220 deployed on one or more enterprise servers includes a drug library editor 222 with a drug library 224. Each of the infusion pumps 240 includes an operational drug library 242, with each of the operational drug libraries 242 for each individual infusion pump 240 being tailored for the patient using each individual infusion pump 240, i.e., the operational drug libraries 242 can be different from each other as required to meet the therapy needs of a particular patient, the intended use of the infusion pump, or the location of the pump or patient. The medication management system 200 can include one or more computers/servers with associated software as required for a particular application.

The medication management system (MMS) 200 can optionally include additional information systems in communication with the medication management unit (MMU) 220 and infusion pumps 240 across the electronic network 230. In various embodiments, the medication management system (MMS) 200 can optionally include a pharmacy information system (PhIS) 218 for delivering drug orders to a hospital information system (HIS) 210, a physician/provider order entry (POE) 216 permitting a healthcare provider to deliver a medication order prescribed for a patient to the hospital information system, a lab system 214 to monitor patient data and to deliver updated patient-specific information such as lab tests, measurements or the like to the MMU 220, and/or a medication administration record system (MAR) 212 for maintaining medication records. The medication management system (MMS) 200 can also include monitoring devices (not shown) in communication with the MMU 220 and/or lab system 214, for monitoring condition of the patient using each of the infusion pumps 240.

The hospital information system (HIS) 210 can be any information system operable to communicate across the hospital environment. The hospital information system 210 can include and/or communicate with the medication administration record system (MAR) 212 to maintain medication records and the pharmacy information system (PhIS) 218 to deliver drug orders to the hospital information system 210. The physician/provider order entry (POE) device 216 can permit a healthcare provider to deliver a medication order prescribed for a patient to the hospital information system 210 directly or indirectly via the PhIS 218. One skilled in the art will also appreciate that a medication order can be sent to the MMU 220 directly from the PhIS 218 or POE device 216. As used herein the term medication order is defined as an order or “prescription” to administer something that has a physiological impact on a person or animal, including but not limited to liquid or gaseous fluids, drugs or medicines, liquid nutritional products and combinations thereof.

The medication management unit 220 includes the drug library editor 222 with a drug library 224. The drug library editor 222 operates on the drug library 224 within the drug library editor 222 to support, maintain, edit, import and/or export the operational drug library received by the infusion pumps 240. A programmer (not shown), such as a licensed pharmacist, doctor, or the like, can work with the drug library editor on a user interface to create and/or modify the drug library 224 as desired for a particular application. The drug library editor 222 can be deployed in the medication management unit 220 on one or more computers and enables the programmer to import, export, and edit whole drug libraries and individual drug library values to control and customize a drug library according to hospital preferences.

The medication management unit 220 can perform other functions in addition to being the drug library editor. In one embodiment, the medication management unit 220 can further maintain patient safety by reviewing medication orders, drug-drug compatibility, and medication delivery time sequences. In another embodiment, the medication management unit 220 can modulate performance of a medication order based on laboratory data or other newly received patient information. In another embodiment, the medication management unit 220 can monitor the status of the infusion pumps and infusion status progress (including alarms, event logs, and pump user interface inputs), generate reports, and control software or operating code updates to the infusion pumps.

The electronic network 230 can be any information network operable to connect the hospital information system (HIS) 210, medication management unit (MMU) 220, and infusion pumps 240. The electronic network 230 can be any combination of wired and/or wireless networks as desired for a particular application. The electronic network 230 can provide communication within a single location, such as a single hospital, or can provide communication over various geographic locations, such as a healthcare network.

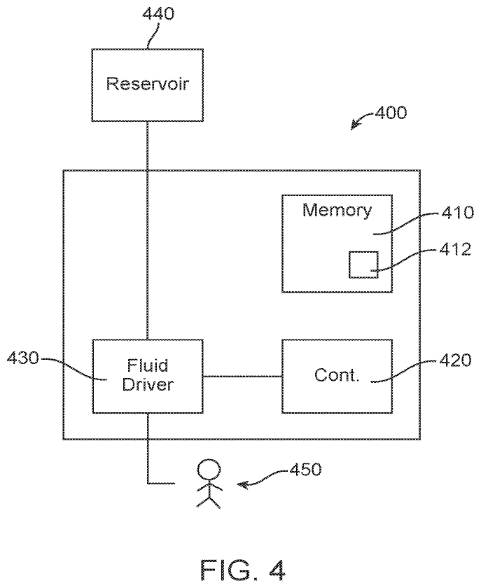

The infusion pumps 240 include one or more infusion pumps with multiple drug library editor source capability. The infusion pumps 240 can be any type of pump, including but not limited to a pump having a pumping mechanism or fluid driver, which acts upon a cassette, reservoir, vial, syringe, or tubing to convey medication or fluid to or from a patient. Exemplary pumps include but are not limited to enteral pumps, infusion pumps, patient controlled analgesia (PCA) or pain management medication pumps, syringe pumps, peristaltic pumps and the like. The pumps could be stationary (pole or bedside mounted and plugged into an AC outlet) or ambulatory (worn or carried by the patient and power by one or more batteries) or some combination of these types or individual features. In one embodiment, the infusion pump with multiple drug library editor source capability can be an infusion pump as described below in association with FIG. 4.

The Hospira MedNet™ safety software available from Hospira, Inc., of Lake Forest, Ill., U.S.A. is one example of software that resides in the medication management unit or MMU 220 of a medication management system 200. The Hospira MedNet™ safety software is described in U.S. patent application Ser. No. 10/930,358 filed Aug. 31, 2004, entitled Medication Management System (now U.S. Pat. No. 7,895,053, issued Feb. 22, 2011), incorporated herein in its entirety by reference. The Hospira MedNet™ safety software links infusion devices with the hospital pharmacy and hospital information systems, enabling clinicians to better bi-directionally communicate, understand and manage IV infusion information with respect to infusion pumps at the point of care. As stated above, one example of an enterprise drug library editor is Hospira MedNet™ Meds™ software. An early version of such software is described in U.S. Pat. No. 8,065,161 to Howard et al., entitled System for Maintaining Drug Information and Communicating with Medication Delivery Devices, which is incorporated herein in its entirety by reference. Using such a drug library editor, hospital pharmacists work in collaboration with a cross-functional medical team to develop customized drug libraries and dose recommendations, which are then programmed into a database and transferred to the infusion pump. The drug libraries can incorporate both hard and soft dose limits, and can customize clinical decision rules for multiple clinical care areas (CCAs). In various applications, the Hospira MedNet™ safety software and Hospira MedNet™ Meds™ drug library editor software can be used with the Hospira Plum A+™ general infusion system, the Hospira Plum A+™ 3 triple-channel device, the Hospira LifeCare PCA™ patient-controlled analgesia system, or the Hospira Symbiq™ infusion pump.

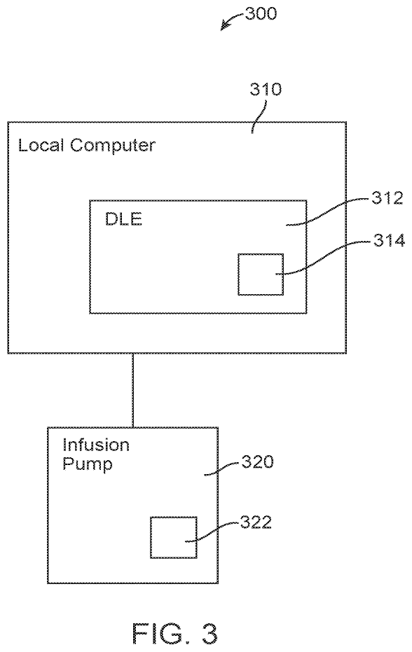

FIG. 3 is a block diagram of an infusion pump system with a standalone, dedicated drug library editor for an infusion pump system with multiple drug library editor source capability in accordance with the present invention. The dedicated drug library editor is a drug library editor operably connectable to and providing a drug library for a single type of infusion pump or a limited set of from one to fifteen infusion pumps of the same type.

The infusion pump system 300 includes a local computer 310 and an infusion pump 320. A dedicated drug library editor 312 having a drug library 314 is deployed on the local computer 310. The infusion pump 320 includes an operational drug library 322. In one embodiment, both the local computer 310 and infusion pump 320 are located at the point of care (POC) of the patient, such as a hospice, nursing home, home care environment, or the like. In another embodiment, the local computer 310 is located in a non-patient care area such as a biomedical engineering area and is used to setup or configure infusion pumps for distribution and use in other locations. In one embodiment, the infusion pump system 300 can provide various exemplary therapies, such as continuous therapy, multistep therapy, intermittent therapy, total parenteral nutrition (TPN), Patient Controlled Analgesia (PCA) therapy, epidural therapy, or the like.

The local computer 310 can be any computing device capable of deploying the dedicated drug library editor 312 as desired for a particular application. Exemplary computing devices include personal computers, tablet computers, personal digital assistants, handheld computers, and the like. The local computer 310 can communicate with the infusion pump 320 when connected to the infusion pump 320 over wired or wireless connections.

The infusion pump 320 is an infusion pump with multiple drug library editor source capability. The infusion pumps 320 can be any type of pump, including but not limited to a pump having a pumping mechanism or fluid driver, which acts upon a cassette, reservoir, vial, syringe, or tubing to convey medication or fluid to or from a patient. Exemplary pumps include but are not limited to enteral pumps, infusion pumps, patient controlled analgesia (PCA) or pain management medication pumps, syringe pumps, peristaltic pumps and the like. In one embodiment, the infusion pump with multiple drug library editor source capability can be an infusion pump as described below in association with FIG. 4.

Exemplary medication management systems with standalone, dedicated drug library editors deployed on or used on a standalone local computer include the GemStar™ SP Infusion System from Hospira, Inc., of Lake Forest, Ill.

FIG. 4 is a block diagram of an infusion pump with multiple drug library editor source capability in accordance with the present invention. The infusion pump can receive an operational drug library from either one of a dedicated drug library editor or an enterprise drug library editor.

The infusion pump 400 is for selective use with either one of a first drug library editor or a second drug library editor as described above. The first drug library editor is operable to generate a first drug library and the second drug library editor is operable to generate a second drug library. One of the first drug library editor and the second drug library editor is a dedicated drug library editor operably connectable to and providing a drug library for a single infusion pump or a single type of infusion pump. The other of the first drug library editor and the second drug library editor is an enterprise drug library editor operably connectable to and providing drug libraries for a large number of infusion pumps or different types of infusion pumps.

The infusion pump 400 includes a memory 410 operable to store an operational drug library 412, a flow controller 420 operably connected to the memory 410, and a fluid driver 430 operably connected to the flow controller 420. The memory 410 is operable to receive one of the first drug library from the first drug library editor and the second drug library from the second drug library editor as the operational drug library 412. The flow controller 420 is operable to control the fluid driver 430 in accordance with the received operational drug library 412 and delivering the infusion to the patient 450. In one embodiment, the infusion pump 400 delivers the infusion from a fluid reservoir 440 to the patient 450. In one embodiment, the fluid driver 430 acts upon a fluid containing device, such as a cassette, reservoir, vial, syringe, tubing, or the like, to convey medication or fluid to or from a patient.

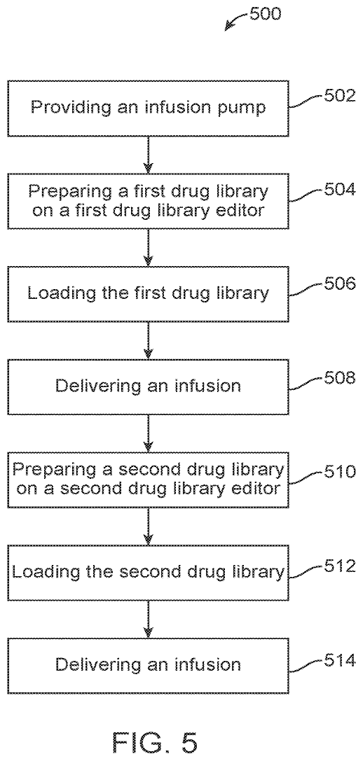

FIG. 5 is a flowchart of a method of use for an infusion pump system with multiple drug library editor source capability in accordance with one embodiment of the present invention. The method can be used with the infusion pump system and infusion pump described above in conjunction with FIG. 1-4.

Referring to FIG. 5, the method 500 includes providing an infusion pump 502 having an operational drug library; preparing a first drug library on a first drug library editor 504; loading the first drug library 506 into the infusion pump from the first drug library editor as the operational drug library; delivering an infusion 508 from the infusion pump in accordance with the first drug library; preparing a second drug library on a second drug library editor 510; loading the second drug library 512 into the infusion pump from the second drug library editor as the operational drug library; and delivering an infusion 514 from the infusion pump in accordance with the second drug library. The first drug library editor is one of a dedicated drug library editor and an enterprise drug library editor, and the second drug library editor is the other of the one of the dedicated drug library editor and the enterprise drug library editor.

In one embodiment, the first drug library editor is the dedicated drug library editor deployed on a local computer operably connectable to and local to the infusion pump. The local computer can be a personal computer, tablet computer, personal digital assistant, handheld computer, or the like. In one embodiment, the delivering an infusion 508 from the infusion pump in accordance with the first drug library further includes delivering the infusion from the infusion pump at a location such as a hospice, a nursing home, a home care environment, or other small treatment location.

In one embodiment, the infusion pump is one of a large number or multitude of infusion pumps or plurality of types of infusion pumps and the first drug library editor is the enterprise drug library editor deployed on a medication management unit operably connectable to and remote from the multitude of infusion pumps or plurality of types of infusion pumps. In one embodiment, the first drug library editor is the enterprise drug library editor deployed on a medication management unit operably connectable to and remote from the infusion pump, and the delivering an infusion 508 from the infusion pump in accordance with the first drug library further includes delivering the infusion from the infusion pump at a location such as a hospital, a treatment center, or other large treatment location.

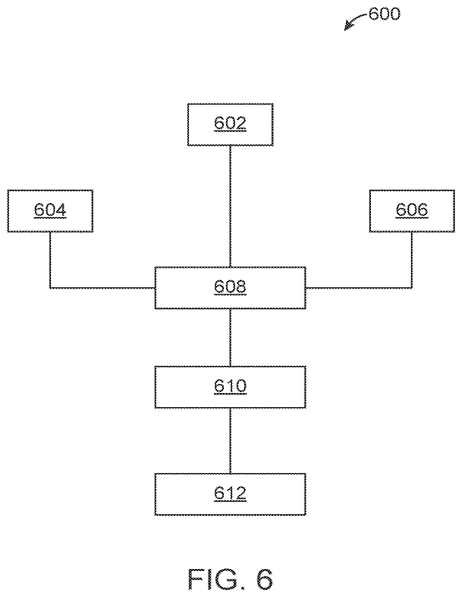

FIG. 6 is a flowchart of another embodiment of a method of use for an infusion pump system with multiple drug library editor source capability in accordance with the present invention. The method can be used with the infusion pump system and infusion pump described above in conjunction with FIG. 1-4.

Referring to FIG. 6, the method 600 includes the step 602 of providing an infusion pump with multiple drug library editor source capability; preparing in step 604 a first drug library using a first drug library editor and/or preparing in step 606 a second drug library using a second drug library editor; selecting in step 608 a source drug library editor from one of the first drug library editor and the second drug library editor; loading in step 610 one of the first drug library and the second drug library into the infusion pump from the selected source drug library editor as an operational drug library; and in step 612 operating the infusion pump in accordance with the loaded operational drug library, in other words using the configuration and operational parameters defined in the operational drug library.

In one embodiment of the method 600 the step 608 of selecting a source drug library editor is performed at the infusion pump by a user making a selection from a plurality of possible selections on a user interface of the pump. In one embodiment, a download drug library function could be facilitated by a pump user interface display screen with a drop-down menu of drug library editor sources. The operational drug library is requested by the infusion pump to be downloaded from the selected source drug library editor. In another embodiment, the selecting of the source drug library editor is performed at one of the first and second drug library editors by a user of the drug library editors. A drug library editor user interface display screen can provide a drop-down menu or other means of selection for the drug library editor source. Then the selected drug library editor source can download the respective drug library to the target pump or pumps. In another embodiment, a special handshake between the drug library editor and the pump could identify the drug library editor source selected at the drug library editor. The download message or drug library itself could contain a code, configuration setting or direction as to where to accept the next drug library from. In yet another embodiment, the selection of the source drug library editor is performed at the medication management unit (MMU) depending on healthcare facility preferences and practices for a given infusion pump or set of infusion pumps.

One advantage of the present invention is that it provides a way to migrate one or more pumps from a dedicated drug library editor system to an enterprise drug library editor system without having to change out the pumps. It is also possible to remove pumps from an enterprise drug library editor environment and place them in a dedicated drug library environment like an alternative care site, nursing home, or patient's home without changing the pump. This seamless repurposing of pumps makes their inventory and associated capital costs easier to manage. Finally, the present invention allows the dedicated drug library editor to be more focused and therefore optimized in its limited functions for the type of pump it serves. The less complex dedicated drug library editor would be less costly to produce (and purchase), easier to setup, understand and run. Conversely, an enterprise drug library editor can be more easily life cycle managed, such as upgraded with bug fixes, new components or additional functionality, pump types, etc. The present invention allows for the leveraging of the advantages of both types of drug library editors in a single pump with multiple drug library editor capability.

While the embodiments of the invention disclosed herein are presently considered to be preferred, various changes, rearrangement of steps, and modifications can be made without departing from the scope of the invention. The scope of the invention is indicated in the appended claims, and all changes that come within the meaning and range of equivalents are intended to be embraced therein.

What is claimed is:

1. A method of configuring one or more infusion pumps comprising:
providing an infusion pump adapted to receive a drug library from one of multiple drug library editors and having an operational drug library;
preparing one of a first drug library on a first drug library editor or a second drug library on a second drug library editor;
selecting one of the first drug library editor and the second drug library editor as a source for a received drug library comprising one of the first drug library and the second drug library;
loading the received drug library into the infusion pump as the operational drug library;
delivering an infusion from the infusion pump in accordance with the operational drug library;
wherein the first drug library editor is one of a dedicated drug library editor and an enterprise drug library editor, and the second drug library editor is the other of the one of the dedicated drug library editor and the enterprise drug library editor.

2. The method of claim 1, wherein selecting one of the first drug library editor and the second drug library editor as the source for the received drug library is performed at the infusion pump through a user selection on a user interface of the infusion pump.

3. The method of claim 1, wherein selecting one of the first drug library editor and the second drug library editor as the source for the received drug library is performed at one of the first drug library editor and the second drug library editor through a user interface of the one of the first drug library editor and the second drug library editor.

4. The method of claim 3, wherein the user interface of the one of the first drug library editor and the second drug library editor includes a drop-down menu for selecting the source for the received drug library.

5. The method of claim 1, wherein selecting one of the first drug library editor and the second drug library editor comprises receiving, at the infusion pump, a download message including an indicator identifying a selection of the operational drug library.

6. The method of claim 5, wherein loading the received drug library into the infusion pump as the operational drug library is performed based on the indicator in the download message.

7. The method of claim 1, wherein selecting one of the first drug library editor and the second drug library editor comprises performing a handshake between the infusion pump and one of the first drug library editor and the second drug library editor.

8. The method of claim 8, wherein the handshake identifies which of the first drug library editor and the second drug library editor is the selected source for the received drug library.

9. The method of claim 1, wherein the received drug library includes a code, configuration setting, or direction indicating where the infusion pump is to accept a next drug library from.

10. The method of claim 1, wherein the operational drug library includes a configuration setting indicating where the infusion pump is to accept a next drug library from.

11. The method of claim 10, wherein the configuration setting enables migration of the infusion pump from receiving drug libraries from the dedicated drug library editor to receiving drug libraries from the enterprise drug library editor.

12. The method of claim 11, wherein the configuration setting enables migration of the infusion pump from receiving drug libraries from the enterprise drug library editor to receiving drug libraries from the dedicated drug library editor.

13. The method of claim 1, wherein the infusion pump is migrated from a dedicated drug library editor system to an enterprise drug library editor system without changing out the infusion pump.

14. The method of claim 1, wherein the infusion pump is removed from an enterprise drug library editor environment and placed in a dedicated drug library editor environment without changing out the infusion pump.

15. The method of claim 1, wherein the dedicated drug library editor provides a protocol-based drug library and the enterprise drug library editor provides a drug profile-based drug library.

16. The method of claim 1, wherein the enterprise drug library editor is configured to provide drug libraries for different types of infusion pumps.

17. The method of claim 16, wherein the different types of infusion pumps differ by at least one of operating mechanism, manufacturer, model, software version, or hardware version.
